(19) United States
(12) Patent Application Publication
(10) Pub. No.: US 2014/0030355 A1
(43) Pub. Date: Jan. 30, 2014

(54) FEED COMPOSITION COMPRISING A MINERAL COMPLEX AND METHODS OF USING THE MINERAL COMPLEX

(71) Applicant: Justin Cannock, Scottsdale, AZ (US)

(72) Inventor: Justin Cannock, Scottsdale, AZ (US)

(21) Appl. No.: 13/968,037

(22) Filed: Aug. 15, 2013

Related U.S. Application Data
(63) Continuation of application No. 13/150,795, filed on Jun. 1, 2011, now abandoned.
(60) Provisional application No. 61/350,386, filed on Jun. 1, 2010.

(51) Int. Cl. A23K 1/175; A61K 45/06; A61K 33/00; A61K 33/26; A61K 33/08

(52) U.S. Cl. CPC A23K 1/175; A61K 33/26; A61K 33/08; A61K 33/00; A61K 45/06
USPC 424/601; 424/647; 424/639

(57) ABSTRACT

The invention provides a mineral complex comprising about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO. The invention also provides animal feeds comprising the mineral complex. Methods for increasing the feed efficiency and weight gain in an animal by administering the mineral complex or an animal feed comprising the mineral complex also are provided.

### CROSS-REFERENCE TO RELATED APPLICATIONS

This patent application claims the benefit of U.S. Provisional Patent Application No. 61/350,386, filed Jun. 1, 2010, the entirety of which is incorporated by reference.

### BACKGROUND OF THE INVENTION

The production of organic foods is based on a system of farming that maintains and replenishes soil fertility without the use of toxic and persistent pesticides and fertilizers. Organically produced foods also must be produced without the use of antibiotics, synthetic hormones, genetic engineering and other excluded practices, such as sewage sludge or irradiation. Organic foods and beverages continue to be one of the fastest growing segments in the overall food market. The fastest growing food categories are organic meat, organic dairy products, and organic fruits and vegetables.

While demand for antibiotic free/organic meat is growing at a rate of approximately 20% per year, the complexities and associated high costs of production remain substantial impediments to the antibiotic free/organic industry. Antibiotic supplementation is routinely used to treat diseases, enhance feed utilization, and to otherwise benefit the health and/or metabolism of food producing animals. The use of antibiotics allows greater production from animals (e.g., in the faun of meat, eggs and milk) from the same quantity of feed, thus allowing greater potential for profitability. Antibiotic free livestock producers must compete in the marketplace with commodity producers, and while the educated consumer understands the real dangers of antibiotic levels in commodity animal products, the higher costs of antibiotic free/organic products deter the consumer's desire for healthful fare.

It has recently been suggested that mineral products can be added to livestock feed compositions to reduce or replace antibiotics. However, many available mineral products contain high levels of minerals and elements that can be toxic to animals. For example, many mineral products comprise levels of heavy metals which have been identified by the EPA and FDA as toxic to humans and animals. In addition, highly purified mineral products are very expensive and further drive up the costs associated with the production of antibiotic free/organic animal products.

In view of the foregoing, there exists a need for a cost-effective, organic, non-toxic, substitute for antibiotics that can be used in livestock feed compositions. The invention described herein overcomes the problems associated with the high cost of antibiotic free/organic animal products by providing a safe and cost-effective mineral complex that can be added to animal feed compositions.

### BRIEF SUMMARY OF THE INVENTION

In one aspect, the invention provides a method for supplementing the diet of an animal in need thereof comprising administering to the animal a palatable amount of a mineral complex, wherein the mineral complex comprises about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO.

The invention also provides a supplemented animal feed comprising an edible animal feed and a palatable amount of a mineral complex, wherein the mineral complex comprises about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO.

The invention provides a method for increasing weight gain in an animal in need thereof comprising administering to the animal a supplemented animal feed comprising an edible animal feed and a palatable amount of a mineral complex, wherein the mineral complex comprises about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO.

The invention further provides a method for increasing milk production in dairy animals in need thereof comprising administering to the animal a supplemented animal feed comprising an edible animal feed and a palatable amount of a mineral complex, wherein the mineral complex comprises about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO.

The invention provides a method for increasing egg production in poultry in need thereof comprising administering to the poultry a supplemented animal feed comprising an edible animal feed and a palatable amount of a mineral complex, wherein the mineral complex comprises about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO.

The invention also provides a process for preparing a supplemented animal feed comprising: (a) providing a prepared or formulated edible animal feed; and (b) adding to the edible animal feed a palatable amount of a mineral complex comprising about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO.

### DETAILED DESCRIPTION OF THE INVENTION

The invention provides a mineral complex that can be administered to animals alone or in combination with various other consumables, as well as methods of using the mineral complex.

The mineral complex of the invention may be obtained from any natural mineral source. In this regard, the mineral complex of the invention is a natural product which may be used for the production of organic foods. In particular, the mineral complex of the invention meets Organic Materials Review Institute (OMRI) certification standards as organic/non-synthesized inputs: natural mineral deposits that are not chemically processed/altered. The mineral complex of the invention also qualifies as Generally Recognized As Safe (GRAS) status for food processing applications.

Desirably, the mineral complex of the invention is obtained from volcanic (mineral) deposits. For example, the mineral deposit may be located in non-porous naturally altered volcanic lava, from highly porous naturally altered volcanic ash, or the mineral complex may be prepared from a combination of non-porous naturally altered volcanic lava and highly porous naturally altered volcanic ash.

The mineral complex of the invention may be obtained from any source that extracts mineral deposits having the characteristics described herein. The mineral complex may be obtained from one source or from several different sources. For example, the mineral complex may be prepared from one natural source of mineral deposit having the characteristics described herein, or the mineral complex can be prepared by mixing together several different mineral deposits to achieve a mineral complex having the characteristics described herein. It should be further understood that when the mineral complex of the invention is obtained from a natural source, the mineral complex may vary in content from source to source and batch to batch. However, analyses of the mineral complex may be routinely performed in accordance with techniques known to those skilled in the art to ensure that the quality of the mineral complex is maintained from batch to batch and from source to source.

The mineral complex of the invention comprises numerous minerals and elements as described herein. Unless otherwise indicated by language or context, references to weight percents of the minerals and elements of which the mineral complex is comprised are based on the total weight of the mineral complex.

The component having the highest weight percent (wt. %) in the mineral complex is silicon dioxide (SiO₂). For example, the mineral complex may comprise about 40 wt. % to about 60 wt. %, about 42 wt. % to about 58 wt. %, about 45 wt. % to about 60 wt. %, about 45 wt. % to about 55 wt. %, about 45 wt. % to about 50 wt. %, about 46 wt. % to about 56 wt. %, about 47 wt. % to about 55 wt. %, about 47 wt. % to about 54 wt. %, about 48 wt. % to about 53 wt. %, or about 47 wt. % to about 49 wt. % SiO₂. Desirably, the mineral complex comprises less than 56 wt. % (e.g., about 45 wt. %, about 46 wt. %, about 47 wt. %, about 48 wt. %, about 49 wt. %, about 50 wt. %, about 51 wt. %, about 52 wt. %, about 53 wt. %, about 54 wt. %, or about 55 wt. %) SiO₂.

The mineral complex of the invention also may comprise an iron oxide (e.g., FeO, Fe₂O₃, or Fe₃O₄). For example, the mineral complex may comprise about 6 wt. % to about 16 wt. %, about 8 wt. % to about 16 wt. % (e.g., about 8 wt. %, about 9 wt. %, about 10 wt. %, about 11 wt. %, about 12 wt. %, about 13 wt. %, about 14 wt. %, about 15 wt. %, or about 16 wt %), about 9 wt. % to about 15 wt. %, about 10 wt. % to about 14 wt. %, about 9 wt. % to about 14 wt. %, about 10 wt. % to about 13 wt. %, about 12 wt. % to about 14 wt. %, or about 12 wt. % to about 16 wt. % Fe₂O₃.

The mineral complex of the invention also may comprise calcium oxide (CaO). For example, the mineral complex may comprise about 4 wt. % to about 12 wt. %., about 6 wt. % to about 12 wt. %., about 6 wt. % to about 11 wt. %. (e.g., about 6 wt. %, about 7 wt. %, about 8 wt. %, about 9 wt. %, about 10 wt. %, about 11 wt. %), about 6 wt. % to about 10 wt. %, about 7 wt. % to about 10 wt. %, or about 7 wt. % to about 9 wt. % CaO.

The mineral complex of the invention also may comprise magnesium oxide (MgO). For example, the mineral complex may comprise about 2 wt. % to about 8 wt. %, about 4 wt. % to about 8 wt. %, (e.g., about 4 wt. %, about 5 wt. %, about 6 wt. %, about 7 wt. %, or about 8 wt. %), about 5 wt. % to about 8 wt. % , about 4 wt. % to about 7 wt. %, or about 5 wt. % to about 6 wt. % MgO.

It is contemplated that the foregoing ranges of each component of the mineral complex may be present in the mineral complex in any combination. For example, the mineral complex may comprise about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO, or the mineral complex may comprise about 40 wt. % to about 60 wt. % SiO₂, about 12 wt. % to about 16 wt. % Fe₂O₃, about 7 wt. % to about 11 wt. %. CaO, and about 2 wt. % to about 8 wt. % MgO. Additional exemplary mineral complexes may comprise about 47 wt. % to about 54 wt. % SiO₂, about 9 wt. % to about 15 wt. % Fe₂O₃, about 6 wt. % to about 10 wt. % CaO, and about 4 wt. % to about 7 wt. % MgO; about 48 wt. % to about 53 wt. % SiO₂, about 10 wt. % to about 14 wt. % Fe₂O₃, about 6 wt. % to about 9 wt. % CaO, and about 4 wt. % to about 6 wt. % MgO; about 49 wt. % to about 53 wt. % SiO₂, about 9 wt. % to about 12 wt. % Fe₂O₃, about 7 wt. % to about 9 wt. % CaO, and about 5 wt. % to about 6 wt. % MgO; or about 47 wt. % to about 49 wt. % SiO₂, about 12 wt. % to about 15 wt. % Fe₂O₃, about 8 wt. % to about 10 wt. % CaO, and about 5 wt. % to about 7 wt. % MgO.

Desirably, the mineral complex comprises about 46 wt. % to about 50 wt. % SiO₂, about 12 wt. % to about 14 wt. % Fe₂O₃, about 8 wt. % to about 10 wt. % CaO, about 5 wt. % to about 7 wt. % MgO, about 14 wt. % to about 16 wt. % Al₂O₃, and about 1 wt. % to about 4 wt. % Na₂O. Preferably, the mineral complex comprises about 47 to about 49 (about 48) wt. % SiO₂, about 13 to about 15 (about 14) wt. % Fe₂O₃, about 8 to about 10 (about 9) wt. % CaO, and about 5 to about 7 (about 6) wt. % MgO. In another embodiment, the mineral complex comprises about 49 to about 51 (about 50) wt. % SiO₂, about 12 to about 14 (about 13) wt. % Fe₂O₃, about 8 to about 10 (about 9) wt. % CaO, and about 5 to about 7 (about 6) wt. % MgO. In yet another embodiment, the mineral complex comprises about 52 to about 54 (about 53) wt. % SiO₂, about 9 to about 11 (about 10) wt. % Fe₂O₃, about 6 to about 8 (about 7) wt. % CaO, and about 4 to about 6 (about 5) wt. % MgO.

The mineral complex of the invention also may further comprise aluminum oxide (Al₂O₃). Preferably, the mineral complex may comprise less than about 16 wt. % Al₂O₃, about 12 wt. % or about 10 wt. %. For example, the mineral complex may comprise about 9 wt. % to about 15 wt. %, about 12 wt. % to about 15 wt. % (e.g., about 12 wt. %, about 13 wt. %, about 14 wt. %, about 15 wt. %) about 13 wt. % to about 15 wt. %, or about 14 wt. % to about 15 wt. % Al₂O₃. The mineral complex of the invention also may further comprise sodium oxide (Na₂O). For example, the mineral complex comprises about 1 wt. % to about 4 wt. %, about 2 wt. % to about 3 wt. % (e.g., about 2 wt. %, about 2.5 wt. %, or about 3 wt. %) Na₂O.

Desirably, the mineral complex also may comprise at least one or more rare earth elements (e.g., at least two, at least three, at least four, or at least five). As defined by IUPAC, rare earth elements (which include for purposes of the invention that which may be referred to as rare earth metals) are a collection of seventeen chemical elements in the periodic table, namely scandium (Sc), yttrium (Y), and the fifteen lanthanoids: lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). The rare earth elements are also referred to as light rare earth elements (lanthanum, cerium praseodymium, neodymium, promethium, and samarium) and heavy rare earth elements (europium, gadolinium, terbium, dysprosium, holmium, erbium, thulium, ytterbium, and lutetium). Scandium and yttrium are considered rare earths since they tend to occur in the same ore deposits as the lanthanoids and exhibit similar chemical properties.

In this regard, the mineral complex may comprise one or more rare earth elements selected from the group consisting of scandium, yttrium, lanthanum, cerium praseodymium, neodymium, promethium, samarium, europium, gadolinium, terbium, dysprosium, holmium, erbium, thulium, ytterbium, and lutetium. It is desirable that the mineral complex comprise at least one light rare earth element and at least one heavy rare earth element. In other embodiments, the mineral complex may comprise only light rare earth elements or only heavy rare earth elements. In a preferred embodiment, the mineral complex may comprise less than 50 ppm of each rare earth element. For example, the mineral complex may comprise about 40 ppm, about 30 ppm, about 25 ppm, about 20 ppm, about 15 ppm, about 10 ppm, about 5 ppm, about 4 ppm, about 3 ppm, about 2 ppm, about 1 ppm, or about 0.5 ppm of one or more rare earth elements. In another embodiment the mineral complex may comprise about 0.5 ppm to about 49 ppm, about 0.5 ppm to about 40 ppm, about 0.5 ppm to about 30 ppm, about 0.5 ppm to about 25 ppm, about 0.5 ppm to about 20 ppm, about 0.5 ppm to about 15 ppm, about 0.5 ppm to about 10 ppm, about 0.5 ppm to about 5 ppm, about 0.5 ppm to about 1 ppm, or about 0.5 ppm of one or more rare earth elements.

In a further embodiment, the mineral complex may comprise one or more of the following oxidized components: potassium oxide (K₂O), chromium oxide (Cr₂O₃), titanium oxide (TiO₂), manganese oxide (MnO), phosphorous oxide (P₂O₅), strontium oxide (SrO), and barium oxide (BaO). For example, the mineral complex may comprise about 0.001 wt. % to about 3 wt. %. about 0.01 wt. % to about 3 wt. %, about 0.01 wt. % to about 2 wt. %, about 0.1 wt. % to about 1 wt. %, or about 0.5 wt. % to about 1 wt. % K₂O, Cr₂O₃, TiO₂, MnO, P₂O₅, SrO, and/or BaO. In one embodiment, the mineral complex comprises less than about 3 wt. % (e.g., about 2.5 wt. %, about 2 wt. %, about 1.5 wt. %, about 1 wt. %, about 0.5 wt. %, or less than 0.5 wt. %) K₂O, Cr₂O₃, TiO₂, MnO, P₂O₅, SrO, and/or BaO. In another embodiment, the mineral complex comprises one or more of the following: about 0.5 wt. % to about 0.9 wt. % K₂O, about 0.01 wt. % to about 0.03 wt. % Cr₂O₃, about 1.0 wt. % to about 2.0 wt. % TiO₂, about 0.1 wt. % to about 0.3 wt. % MnO, about 0.1 wt. % to about 0.3 wt. % P₂O₅, about 0.01 wt. % to about 0.05 wt. % SrO, and/or about 0.01 wt. % to about 0.03 wt. % BaO.

The mineral complex of the invention also may comprise carbon (C). Preferably, the mineral complex comprises less than about 2 wt. % C. For example, the mineral complex may comprise about 1.5 wt. %, about 1.0 wt. %, about 0.5 wt. %, about 0.25 wt. %, about 0.1 wt. %, about 0.05 wt. %, about 0.04 wt. %, about 0.03 wt. %, or less than about 0.03 wt. % C. The mineral complex of the invention also may comprise sulfur (S). Preferably, the mineral complex comprises less than 1% S. For example, the mineral complex may comprise about 0.9 wt. %, about 0.5 wt. %, about 0.25 wt. %, about 0.1 wt. %, about 0.05 wt. %, about 0.04 wt. %, about 0.03 wt. %, about 0.02 wt. %, about 0.01 wt. %, or less than about 0.01 wt. % S.

The mineral complex of the invention also may further comprise at least one or more of (e.g., at least two or more of, at least three or more of, at least four or more of, or at least five or more of) the following: silver (Ag), barium (Ba), cobalt (Co), chromium (Cr), caesium (Cs), copper (Cu), gallium (Ga), hafnium (Hf), molybdenum (Mo), niobium (Nb), nickel (Ni), lead (Pb), rubidium (Rb), tin (Sn), strontium (Sr), tantalum (Ta), thorium (Th), thallium (Tl), uranium (U,) vanadium (V), tungsten (W), zinc (Zn), and zirconium (Zr). When present, the mineral complex preferably comprises less than 1000 ppm each of Ag, Ba, Co, Cr, Cs, Cu, Ga, Hf, Mo, Nb, Ni, Pb, Rb, Sn, Sr, Ta, Th, Tl, U, V, W, Zn, or Zr. For example, the mineral complex may comprise one or more of Ag, Ba, Co, Cr, Cs, Cu, Ga, Hf, Mo, Nb, Ni, Pb, Rb, Sn, Sr, Ta, Th, Tl, U, V, W, Zn, and Zr in the ranges set forth in Table 1.

TABLE 1

| | Range |
|---|---|
| | (ppm based on total weight |
| Element | of mineral complex) |
| Ag | about 0.0001 ppm to about 10 ppm |
| Ba | about 1 ppm to about 500 ppm |
| Co | about 0.01 ppm to about 200 ppm |
| Cr | about 1 ppm to about 400 ppm |
| Cs | about 0.0001 ppm to about 50 ppm |
| Cu | about 1 ppm to about 400 ppm |
| Ga | about 0.001 ppm to about 200 ppm |
| Hf | about 0.001 ppm to about 50 ppm |
| Mo | about 0.001 ppm to about 25 ppm |
| Nb | about 0.001 ppm to about 100 ppm |
| Ni | about 1 ppm to about 400 ppm |
| Pb | about 0.001 ppm to about 25 ppm |
| Rb | about 1 ppm to about 400 ppm |
| Sn | about 0.0001 ppm to about 15 ppm |
| Sr | about 1 ppm to about 600 ppm |
| Ta | about 0.0001 ppm to about 10 ppm |
| Th | about 0.001 ppm to about 200 ppm |
| Tl | about 0.0001 ppm to about 25 ppm |
| U | about 0.0001 ppm to about 25 ppm |
| V | about 1 ppm to about 600 ppm |
| W | about 0.0001 ppm to about 25 ppm |
| Zn | about 1 ppm to about 400 ppm |
| Zr | about 1 ppm to about 400 ppm |

The mineral complex of the invention also may comprise one or more of (e.g., one, two, three, four, five, or all six of): arsenic (As), bismuth (Bi), mercury (Hg), antimony (Sb), selenium (Se), and tellurium (Te). Preferably, the mineral complex comprises less than 5 ppm of As, less than 5 ppm of Bi, less than 1 ppm of Hg, less than 5 ppm of Sb, less than 5 ppm of Se, and/or less than 5 ppm of Te. For example, the mineral complex may comprise one or more of As, Bi, Hg, Sb, Se, and Te in the ranges set forth in Table 2.

TABLE 2

| | Range |
|---|---|
| | (ppm based on total weight |
| Element | of mineral complex) |
| As | about 0.0001 ppm to about 4 ppm |
| Bi | about 0.0001 ppm to about 4 ppm |
| Hg | about 0.00001 ppm to about 0.5 ppm |
| Sb | about 0.0001 ppm to about 2 ppm |
| Se | about 0.0001 ppm to about 4 ppm |
| Te | about 0.0001 ppm to about 2 ppm |

In one embodiment, the mineral complex described herein has one or more (e.g., at least two, at least three, at least four, or at least five) of the following characteristics: an average weight in tons per cubic yard of about 1 to about 1.5 (e.g., about 1.3); a loss on ignition of about 0.2% to about 0.3% (e.g., about 0.25%); a fusion of about 2100 to about 2300 degrees Fahrenheit (e.g., about 2200); a mill abrasion loss (A.R.E.A.) of about 5% to about 6% (e.g., about 5.4%); a L.A. abrasion loss according to ASTM C535-89 of about 7.2 to about 8.2 (e.g., about 7.7); a L.A. abrasion loss according to ASTM C 131-89 of about 10.3 to about 11.3 (e.g., about 10.8); a specific gravity according to ASTM C97 of about 2.900 to about 3.060 (e.g., about 2.980); a specific gravity according to ASTM C 127 of about 2.900 to about 3.060 (e.g., about 2.980); an absorption according to ASTM C 127 of less than about 0.5% (e.g., less than 0.4%); and/or a soundness loss according to ASTM C 88 Mg Su of about 0.25% to about 0.75% (e.g., about 0.5%).

The mineral complex of the invention may be obtained in any physical size. Preferably, however, the mineral complex is crushed, ground, and/or milled into a powdered form using any routine methods known in the art.

In one embodiment, the mineral complex is in a powdered form wherein the particles have an average particle size of about 10 to about 6000 mesh. In one embodiment, the particles are very fine and have an average particle size of about 400 to about 6000 mesh, which corresponds to a size of about 37 microns to about 1 micron. For example, and in this embodiment, the particles may have an average particle size of about 400 to about 6000 mesh, about 400 to about 5000 mesh, about 400 to about 4000 mesh, about 400 to about 3000 mesh, about 400 to about 2000 mesh, about 400 to about 1000 mesh, about 400 to about 900 mesh, about 400 to about 800 mesh, about 400 to about 700 mesh, about 400 to about 600 mesh, about 400 to about 500 mesh, about 500 to about 6000 mesh, about 600 to about 6000 mesh, about 700 to about 6000 mesh, about 800 to about 6000 mesh, about 900 to about 6000 mesh, about 1000 to about 6000 mesh, about 2000 to about 6000 mesh, about 3000 to about 6000 mesh, about 4000 to about 6000 mesh, about 5000 to about 6000 mesh, about 500 to about 5000 mesh, about 600 to about 4000 mesh, about 700 to about 3000 mesh, about 800 to about 2000 mesh, about 900 to about 1000 mesh, about 1000 to about 6000 mesh, about 1500 to about 5500 mesh, about 2500 to about 5000 mesh, about 3000 to about 4500 mesh, about 3500 to about 4000 mesh, or about 4000 to about 6000 mesh.

In a related embodiment, the particles are slightly larger, having an average particle size of about 200 to about 400 mesh, which corresponds to a size of about 74 microns to about 37 microns. For example, and in this embodiment, the particles may have an average particle size of about 200 to about 400 mesh, about 230 to about 400 mesh, about 250 to about 400 mesh, about 275 to about 400 mesh, about 300 to about 400 mesh, about 325 to about 400 mesh, about 350 to about 400 mesh, about 375 to about 400 mesh, about 200 to about 375 mesh, about 200 to about 350 mesh, about 200 to about 325 mesh, about 200 to about 300 mesh, about 200 to about 275 mesh, about 200 to about 250 mesh, about 200 to about 225 mesh, or about 250 to about 350 mesh.

In yet another related embodiment, the particles have an even larger particle size of about 10 to about 200 mesh, which corresponds to a size of about 2000 microns to about 74 microns. For example, and in this embodiment, the particles can have an average particle size of about 10 to about 200 mesh, about 20 to about 200 mesh, about 40 to about 200 mesh, about 60 to about 200 mesh, about 80 to about 200 mesh, about 100 to about 200 mesh, about 120 to about 200 mesh, about 140 to about 200 mesh, about 160 to about 200 mesh, about 180 to about 200 mesh, about 10 to about 180 mesh, about 10 to about 160 mesh, about 10 to about 140 mesh, about 10 to about 120 mesh, about 10 to about 100 mesh, about 10 to about 80 mesh, about 10 to about 60 mesh, about 10 to about 40 mesh, about 10 to about 20 mesh, about 40 to about 180 mesh, about 60 to about 160 mesh, or about 80 to about 140 mesh. In a further related embodiment, the particles have an average particle size of about 50 to about 400 mesh, about 100 to about 400 mesh, about 120 to about 400 mesh, about 140 to about 400 mesh, or about 170 to about 400 mesh. The conversion of mesh sizes to microns is well known in the art.

The mineral complex described herein is useful in a variety of different applications, as described herein. Upon referring to the disclosure provided herein, one skilled in the art will appreciate that the particle size of the mineral complex may be selected based on the type of application in which the mineral complex is being used. In addition, one skilled in the art upon reading this disclosure should also appreciate that mineral complex having a relatively smaller average particle size (e.g., a mesh size of about 400 to about 6000 or a mesh size of about 200 to about 400) distributes/suspends/dissolves more readily in any feed composition, such as in grain, commercial feed, water, and the like, as compared to mineral complex having a relatively larger average particle size (e.g., a mesh size of about 10 to about 200).

In addition, one skilled in the art should also appreciate that in some embodiments of the invention it is desirable to use a mineral complex comprising a variety of different average particle sizes. In this regard, it is contemplated that the mineral complex may comprise any combination of the foregoing ranges of particle sizes. For example, the mineral complex (and compositions thereof) described herein may comprise a certain amount of mineral complex having an average particle size of about 400 to about 6000 mesh, and/or a further amount of mineral complex having an average particle size of about 200 to about 400 mesh, and/or an additional amount of mineral complex having an average particle size of about 10 to about 200 mesh. By way of further example, the mineral complex (and compositions thereof) may comprise about 5% to about 85% (e.g., about 5%, about 10%, about 15%, about 20%, about 25%, about 30%, about 35%, about 40%, about 45%, about 50%, about 55%, about 60%, about 65%, about 70%, about 75%, about 80%, or about 85%) of the mineral complex having an average particle size of about 400 to about 6000 mesh, and/or about 15% to about 95% (e.g., about 15%, about 20%, about 25%, about 30%, about 35%, about 40%, about 45%, about 50%, about 55%, about 60%, about 65%, about 70%, about 75%, about 80%, about 85%, about 90%, or about 95%) of the mineral complex having an average particle size of about 200 to about 400 mesh, and/or about 5% to about 75% (e.g., about 5%, about 10%, about 15%, about 20%, about 25%, about 30%, about 35%, about 40%, about 45%, about 50%, about 55%, about 60%, about 65%, about 70%, or about 75%) of the mineral complex having an average particle size of about 10 to about 200 mesh. For example, the mineral complex (and compositions thereof) described herein may comprise about 50% of mineral complex having an average particle size of about 400 to about 6000 mesh and about 50% of mineral complex having an average particle size of about 200 to about 400 mesh, or the composition may comprise about 30% of mineral complex having an average particle size of about 400 to about 6000 mesh, about 50% of mineral complex having an average particle size of about 200 to about 400 mesh, and about 20% of mineral complex having an average particle size of about 10 to about 200 mesh.

Although average particle size may be conveniently measured by scanning electron microscopy (SEM) in accordance with techniques known to those skilled in the art, other techniques also may be used. Preferably, at least 75%, at least 80%, at least 85%, at least 90%, at least 95%, at least 96%, at least 97%, at least 98%, or at least 99% of the particles have a particle size falling within the ranges described herein.

In an alternative embodiment, the mineral complex may be synthetically produced. However, it is preferable to obtain the mineral complex of the invention from a natural source in order to avoid the high cost of synthetically manufacturing the mineral complex of the invention.

The invention also provides methods of using the mineral complex described herein. These methods comprise administering to an animal a palatable amount of a mineral complex as described herein. The mineral complex may be administered orally as part of a lick, or in cubes, or in powdered form, or in oral boluses mixed into flavoring substances or liquid feed (such as molasses), or as a solution of mineral complex that is sprayed onto or mixed into the animals' feed or mixed into the animals' water, or as dry form of mineral complex (e.g., powdered form, tablets, cubes, or pellets) that is mixed into the animals' feed or mixed into the animals' water. In one embodiment of the invention, the mineral complex is fed directly to an animal in dry form. The dry form of the mineral complex may consist of the mineral complex alone, for example, in powdered form. Alternatively, the dry form of the mineral complex may comprise suitable binding agents, such as pellet binders and water stability binders, anti-bridging agents, pellet and extrusion lubricants, additives, stabilizers, and/or excipients known in the art, which may be added to the mineral complex in order to form a tablet, a pellet, a cube, an extruded animal feed, or other suitable form of the mineral complex that can be administered directly to the animal.

In one embodiment, the mineral complex comprises a binding agent. Upon referring to the disclosure provided herein, one skilled in the art will appreciate that a variety of materials may constitute a suitable binder, including an organic material or a synthetic material. For example, the binder may be brewers condensed soluble, wheat powder, beet syrup, molasses, such as beet molasses, desugared beet molasses, or cane molasses, honey, whey, starch, gelatin, sodium casein, sulfur, wax, polymer, oil, urea-formaldehyde, plant starches, protein gels, glues, gumming compositions, seaweed, peat, humic, crystallizing compounds, gelling clays, synthetic gel-forming compounds, and mixtures thereof. Additional examples of binders that may be used herein include carbohydrates, such as monosaccharides, disaccharides, oligosaccharides, and polysaccharides; proteins; lipids; glycolipid; glycoprotein; lipoprotein; and combinations and derivatives of the same. Exemplary carbohydrate binders include glucose, mannose, fructose, galactose, sucrose, lactose, maltose, xylose, arabinose, trehalose, and mixtures thereof, such as corn syrup; celluloses, such as carboxymethylcellulose (CMC), ethylcellulose, hydroxyethylcellulose, hydroxy-methylethylcellulose, hydroxyethylpropylcellulose, methylhydroxyethyl-cellulose, and methylcellulose; starches, such as amylose, seagel, alpha-starch, carboxy-alpha-starch, starch acetates, starch hydroxyethyl ethers, ionic starches, long-chain alkyl starches, dextrins, amine starches, phosphates starches, and dialdehyde starches; plant starches, such as corn starch and potato starch; other carbohydrates, such as pectin, amylopectin, xylan, glycogen, agar, alginic acid, phycocolloids, chitin, gum arabic, guar gum, gum karaya, gum tragacanth, and locust bean gum; complex organic substances, such as lignin and nitrolignin; derivatives of lignin, such as lignin sulfonate and lignosulfonate salts, including calcium lignosulfonate and sodium lignosulfonate; and complex carbohydrate-based compositions containing organic and inorganic ingredients such as molasses. Suitable protein binders include, for example, soy extract, zein, protamine, collagen, and casein. Binders operative herein also include synthetic organic polymers, such as oxide polymers, polyacrylamides, polyacrylates, polyvinyl pyrrolidone, polyethylene glycol, polyvinyl alcohol, polyvinylmethyl ether, polyvinyl acrylates, and polylactic acid.

The composition comprising the binder and mineral complex may be combined by any suitable method, including by subjecting these materials to steam, water, and/or pressure in order to facilitate the agglomeration of the mineral complex and the binder. Suitable methods, such as pelleting and extruding, are well-known in the art. The binder is desirably present in an amount sufficient to provide for the agglomeration of the amount of mineral complex to be processed. For example, the composition may comprise from about 0.1 wt. % to about 99.5 wt. % of the binder (e.g., about 0.1 wt. %, about 0.5 wt. %, about 1 wt. %, about 2 wt. %, about 3 wt. %, about 4 wt. %, about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, about 96 wt. %, about 97 wt. %, about 98 wt. %, about 99 wt. %, or about 99.5 wt. % binder). Desirably, the composition comprises from about 0.1 wt. % to about 50 wt. % binder (e.g., from about 0.1 wt. % to about 5 wt. %, from about 0.5 wt. % to about 10 wt. %, from about 1 wt. % to about 20 wt. %, from about 5 wt. % to about 25 wt. %, or from about 10 wt. % to about 50 wt. % binder). More desirably, the composition comprises from about 0.5 wt. % to about 2.5 wt. % binder (e.g., from about 0.5 wt. % to about 1 wt. %, from about 0.5 wt. % to about 1.5 wt. %, from about 0.5 wt. % to about 2 wt. %, from about 1 wt. % to about 2.5 wt. %, from about 1.5 wt. % to about 2.5 wt. %, from about 2 wt. % to about 2.5 wt. %, or from about 1 wt. % to about 2 wt. %).

Preferably, the binder binds the mineral complex into a form which resists attrition and will not rapidly degrade, and therefore substantially maintains particle size during handling. If desired, the binder may be added to the mineral complex as a solution. The solution is typically provided as a water-based slurry having about 40 to about 50 percent solids by weight and weighing about 10 pounds per gallon. The binder also may be added and mixed with the mineral complex as a dry ingredient, subsequently mixing in an amount of water. In addition, the composition may comprise agents such as anti-bridging agents, pellet and extrusion lubricants, additives, stabilizers, and/or excipients known in the art.

The mineral complex also may be fed to an animal in the form of a lick, which can be prepared using routine methods known in the art. For example, the mineral complex may be mixed with molasses, and then heated and/or compressed to create a block, cube, or pellet, which is then placed in a location accessible to the animal.

Preferably, the mineral complex is administered in either the animal's feed or water. In one embodiment of the invention, the mineral complex is crushed, ground, and/or milled in order to facilitate the addition of the mineral complex to the animal's feed or water, as well as to facilitate consumption and digestion of the mineral complex by the animal. In this regard, the invention provides a supplemented animal feed comprising a prepared or formulated edible animal feed to which has been added a palatable amount of a mineral complex as described herein.

As used herein, a supplemented animal feed can comprise either an admixture of animal feed and the mineral complex or a pre-mixed animal feed. In this regard, the invention contemplates an admixture of animal feed and mineral complex, wherein a palatable amount of the mineral complex described herein is added to and admixed with the final form of the prepared or formulated animal feed. Alternatively, the mineral complex described herein may be added as an ingredient in the animal feed prior to or during processing, such that the resulting final form of the animal feed comprises the mineral complex. For example, for animal feeds that are supplied as pellets (i.e., agglomerated animal feed), the mineral complex can be added as an ingredient in the animal feed prior to pelletization, such that the resulting animal feed pellets comprise the mineral complex incorporated within each pellet, and thus, forms a pre-mixed supplemented animal feed. For extruded animal feed, the mineral complex can be added as an ingredient in the animal feed prior to the extrusion process (e.g., cooking extrusion or forming extrusion), such that the mineral complex is incorporated within the resulting extruded animal feed, and thus, forms a pre-mixed supplemented animal feed. Alternatively, extruded or pelleted animal feed can be obtained and a palatable amount of the mineral complex can be mixed with the pelleted or extruded animal feed to form an admixture of supplemented animal feed.

Animals that could benefit from supplementation in accordance with the invention include any warm or cold blooded animals, including but not limited to mammals, such as cattle (i.e., cows), sheep, goats, swine (e.g., piglets, pigs, and hogs), horses, donkeys, llamas, bison, etc., poultry, such as chickens, turkeys, quail, ducks, geese, etc., fish, moluscs, and crustaceans, such as salmon, catfish, tilapia, cod, koi, carp, trout, goldfish, shrimp, etc., and pets of all kinds , such as dogs, cats, ferrets, birds, and other pets, such as hamsters, gerbils, mice, rats, other rodents and the like. In one embodiment, animals that could benefit from supplementation in accordance with the invention include felines (e.g., lions, tigers, jaguars, and cats), canines (e.g., jackals, wolves, foxes, coyotes, and dogs), fish (e.g., salmon, catfish, tilapia, cod, koi, carp, trout, and goldfish), poultry (e.g., chickens, turkeys, quail, ducks, emus, ostriches, rheas, and geese), cattle, sheep, goats, equines (e.g., horses, donkeys, and zebras), swine, primates (e.g., monkeys, apes, gorillas, chimpanzees, and orangutans), reptiles (e.g., snakes, iguanas, geckos, lizards, turtles, alligators, and crocodiles), birds (e.g., parrots, pigeons, doves, cockatiels, cockatoos, parakeets, and canaries), rabbits, hamsters, gerbils, guinea pigs, mice, rats, giraffes, elephants, bears, camels, hippopotamuses, penguins, and rhinoceroses. Desirably, the animal is a cat, a dog, a chicken, a turkey, a cow, a pig, or a horse.

The feeds into which the mineral complex may be introduced are well known in the art and such feeds are widely available commercially. There are several feed companies that supply feeds specifically formulated for the type of animal being fed. The mineral complex described herein can be added to any commercially available formulation. Alternatively, pet owners, farmers, ranchers, or feedlot operators can prepare feeds suitable for their animals. For cattle and other livestock, suitable feeds are generally mixtures of well-known cereal grains or forage crops, and may have added vitamin and/or mineral supplements.

When added to the animal's feed, the form of the feed must be taken into account. The mineral complex of the invention should be mixed into the feed in the most practical way, and if the animal's diet is partly mixed grains and partly fodder, such as hay, the mineral complex should generally be added to the mixed grain portion. For example, feedlot cattle are generally fed a mixture of grain, grass hay and alfalfa hay, and it is preferable for simplicity, although not essential, to add the mineral complex to the grain portion of the feed. Alfalfa hay is frequently fed in pellet form, and the mineral complex can be incorporated into the alfalfa pellets to provide a pre-mixed supplemented animal feed or the mineral complex can be admixed with the alfalfa pellets. Dairy cattle are often fed a mixture of grain, alfalfa hay and silage, and the mineral complex is most conveniently added to the grain portion of the diet. Pets, such as dogs and cats, are commonly fed commercially available dry or canned pet food, and the mineral complex either can be incorporated into the dry or canned pet food to provide a pre-mixed supplemented animal feed or the mineral complex can simply be admixed with the dry or canned pet food. In general, fish are fed prepared dry fish foods that most commonly are produced in flake, pellet, extruded, or tablet form. The mineral complex either can be incorporated into the dry fish food to provide a pre-mixed supplemented fish food or the mineral complex can be admixed with the dry fish food.

In accordance with the invention, the mineral complex is administered to an animal in an effective amount. In one embodiment, the dosage may be expressed as the amount of mineral complex per pound of animal being supplemented. Alternatively, when the mineral complex is added to animal feed, the amount to be administered may be expressed as the weight of mineral complex per pound of animal feed. Likewise, the concentration of the supplement may be expressed as the amount of mineral complex per pound of animal feed. Different animals, such as fish, dogs, cats, cattle, swine, or poultry, will require administration of the mineral complex at different rates. The mineral complex is administered in an effective amount according to the weight of the animal ingesting the mineral complex. The mineral complex may be used in the feed or water at concentrations below that at which the feed or water becomes unpalatable to the animal.

In accordance with the invention, the mineral complex is supplemented to animals at a rate below that at which the mineral complex decreases the palatability of the food and decreases food intake. Thus, the mineral complex is administered in a palatable concentration or amount. This concentration or amount will of course vary with the type and palatability of the feed being supplemented, and the concentration or amount of the mineral complex administered to the animal may need to be adjusted accordingly. Such adjustments of the supplementation rate are within the skill of the person of ordinary skill in the art. For example, the mineral complex may be administered to animals at a dosage of about 0.01 to about 50 grams per pound animal weight per day, desirably from about 0.01 to about 25 grams per pound animal weight per day, more desirably from about about 0.01 to about 12 grams per pound animal weight per day, and even more desirably from about 0.01 to about 5 grams per pound animal weight per day, and most desirably from about 0.01 to about 1 gram gram per pound animal weight per day.

In one embodiment of the invention, a flavoring agent may be used to increase the palatability of the mineral complex. For example, a flavoring agent can be added to and/or mixed with the mineral complex described herein to provide a flavored mineral complex. The flavored mineral complex can then be fed directly to an animal or, alternatively, the flavored mineral complex can be added to an edible animal feed. Suitable flavoring agents that may be added to the mineral complex are well known in the art and such flavoring agents are widely available commercially. In particular, there are several companies that supply flavoring agents specifically formulated for the type of animal being fed.

In addition, one of ordinary skill in the art will recognize that excess minerals in the diet and water of animals can have an adverse effect on animal health. In this regard, the National Academies convened a committee to make recommendations on animal tolerances and toxic dietary levels, updating a 1980 report on mineral tolerance in domestic animals. Based on a review of current scientific data and information, the report sets a “maximum tolerable level” (MTL) for each mineral as it applies to the diets of farm animals, poultry, and fish. The report includes an analysis of the effects of toxic levels in animal diets, and it identifies elements that pose potential human health concerns. Accordingly, the rate of administration of the mineral complex should be adjusted such that each mineral in the mineral complex does not exceed the MTL for the particular animal being fed. The specific guidelines regarding the MTLs of minerals for animal feed can be found in Mineral Tolerance of Animals, National Research Council of the National Academies, Second Revised Edition, 2005, which is incorporated by reference herein. Such adjustments of the supplementation rate are within the skill of the person of ordinary skill in the art.

In one embodiment, the invention provides a process for preparing a supplemented animal feed comprising: (a) providing a prepared or formulated edible animal feed; and (b) adding to the edible animal feed a palatable amount of a mineral complex as described herein. For example, the palatable amount of the mineral complex may be added to and admixed with the final form of the prepared or formulated animal feed. Alternatively, the mineral complex may be added as an ingredient in the prepared or formulated animal feed prior to or during processing, such that the resulting final form of the prepared or formulated animal feed comprises the mineral complex. Any suitable amount of mineral complex can be added to the prepared or formulated edible animal feed. Preferably, the amount of mineral complex added to the prepared or formulated edible animal feed is less than 20 pounds mineral complex per 100 pounds of the supplemented animal feed. For example, about 0.1 pounds to about 19 pounds, about 0.1 pounds to about 15 pounds, about 0.1 pounds to about 10 pounds, about 0.1 pounds to about 5 pounds, about 0.1 pounds to about 4 pounds, about 0.1 pounds to about 3 pounds, about 0.1 pounds to about 2 pounds, or about 0.1 pounds to about 1 pounds of the mineral complex described herein may be added per 100 pounds of supplemented animal feed. In one embodiment, about 0.5 pounds to about 2.5 pounds, about 1 pounds to about 2 pounds, about 0.5 pounds, about 1 pounds, about 1.5 pounds, about 2 pounds, or about 2.5 pounds of mineral complex may be added per 100 pounds of the supplemented animal feed.

The aforementioned process for preparing a supplemented animal feed produces a supplemented animal feed comprising a mineral complex as described herein in an amount ranging from about 0.1 pounds to about 19 pounds, about 0.1 pounds to about 15 pounds, about 0.1 pounds to about 10 pounds, about 0.1 pounds to about 5 pounds, about 0.1 pounds to about 4 pounds, about 0.1 pounds to about 3 pounds, about 0.1 pounds to about 2 pounds, or about 0.1 pounds to about 1 pounds per 100 pounds of supplemented animal feed. In one embodiment, the supplemented animal feed comprises a mineral complex as described herein in an amount of about 0.5 pounds to about 2.5 pounds, about 1 pounds to about 2 pounds, about 0.5 pounds, about 1 pounds, about 1.5 pounds, about 2 pounds, or about 2.5 pounds per 100 pounds of the supplemented animal feed.

Without being bound by theory, it is thought that providing the mineral complex of the invention to the digestive tract of animals (from ingestion of the mineral complex) will alter the microbial population of the gastrointestinal tract by killing or inhibiting/reducing the growth of the microbes or by altering the microbial metabolism. In addition, the mineral complex may kill or inhibit/reduce the growth of pathogenic organisms in the digestive tract of the animal. Exemplary pathogenic organisms include, but are not limited to, Clostridium species, including Clostridium chauvoei which causes blackleg, Clostridium botulinum which causes botulism, and Clostridium tetani which causes tetanus, Clostridium perfringens which causes necrotic enteritis, Listeria species which cause listeriosis, Nocardia species which cause nocardiosis, Bacillus species, including Bacillus anthracis which causes anthrax, Mycobacterium tuberculosis, which causes bovine tuberculosis, and various species of Streptococcus. Further organisms that may be susceptible to the activity of the mineral complex include those responsible for causing mastitis in dairy cattle, sheep and goats, such as Staphylococcus aureus, Streptococcus uberis, Streptococcus agalactiae, and Streptococcus dysgalactiae. Actinomyces pyogenes, Pseudomonas and other infections, which are less prevalent, may also be prevented or reduced in incidence through the use of the present invention. As another example, protozoa, such as Eimeria species, e.g., E. tenella, E. necatrix, E. acervulina and E. maxima, also may be susceptible to the activity of the mineral complex.

Thus, the invention further provides a method of altering the microbial population of the gastrointestinal tract of an animal in need thereof comprising administering to the animal the mineral complex described herein or an animal feed supplemented with the mineral complex described herein. The method of altering the microbial population of the gastrointestinal tract of an animal in need thereof may include altering the microbial population of the gastrointestinal tract of an animal by inhibiting the growth of microorganisms in the digestive tract of an animal. The microorganisms that may be altered include pathogens. In one embodiment, the microorganisms can be Clostridium species, Listeria species, Nocardia species, Bacillus species, Mycobacterium species, Streptococcus species, Staphylococcus species, Actinomyces species, Pseudomonas species, and Eimeris species.

Because of the activity of the mineral complex, when animals, such as fish, cats, dogs, swine, and cattle, are fed the mineral complex, the animals should show better health, vigor, weight gain, and freedom from diseases. The administration of the mineral complex should also decrease the incidence of conditions such as mastitis and bovine tuberculosis in dairy cattle, and anthrax in beef cattle. These results are similar to the goals achieved by the traditional practice of adding antibiotics to animal feed.

In addition, beef cattle that have ingested the mineral complex should have an altered digestive pattern. Cattle having been fed the mineral complex should be able to ingest a much higher level of molasses before they begin to pass undigested grain. Livestock feeders like to include a high level of molasses in animal feed because it is a cheap source of carbohydrate. However, it has been observed that high amounts of molasses cause the cattle to pass undigested grain. The administration of the mineral complex would be expected to ameliorate or alleviate this type of problem.

Thus, in view of the beneficial effects of the mineral complex of the present invention, the administration of the mineral complex of the invention to animals enhances weight gain in animals, as demonstrated in Example 2 described herein. In particular, when animals ingest the mineral complex, they demonstrate a faster rate of weight gain when compared to animals not ingesting the mineral complex. Without being bound by theory, it is believed that the mineral complex is broken down by the gastrointestinal tract of animals to provide a complete suite of micro and macro minerals that are not normally present in animal feed. As such, the administration of the mineral complex causes the animals to remain healthier relative to animals not being supplemented, thereby allowing them to gain weight easier and quicker. Also, it is believed that the presence of the mineral complex in the gastrointestinal tract of animals alters the microbial population and inhibits the growth of undesirable pathogenic organisms in the tract. Thus, the food nutrients ingested by the animal are more efficiently directed to growth of the animal. The invention, therefore, also provides a method for increasing weight gain in animals. This method comprises administering the mineral complex described herein to the animals. When the animal ingests the mineral complex, the presence of the mineral complex in the animal's digestive tract provides for an increase in weight gain over the weight gain in animals not ingesting the mineral complex. For example, the animal ingesting the mineral complex may exhibit about 1% to about 10% (e.g., about 1%, 2%, 3%, 4%, 5%, 6%, 7%, 8%, 9%, 10%), or more increase in weight gain as compared to the weight gain in animals not ingesting the mineral complex (i.e., animals ingesting animal feed that does not comprise the mineral complex). The animals are preferably fish, beef cattle, dairy cattle, sheep, swine, horses, or poultry.

Because animals ingesting the mineral complex receive additional macro and micro minerals, stay healthier, and because of putative alterations in the makeup of the gastrointestinal flora, there is an increase in the efficiency with which animals convert food to meat, milk, and eggs. Thus, the invention also provides a method for increasing feed efficiency in animals. This method comprises feeding palatable concentrations of the mineral complex described herein to the animals. When the animal ingests the mineral complex, the presence of the mineral complex in the animal's digestive tract provides an increase in feed efficiency over feed efficiency in animals not ingesting the mineral complex. The animals are preferably fish, beef cattle, dairy cattle, sheep, swine, horses or poultry.

When dairy animals are fed the mineral complex of the invention, they exhibit an increase in milk production. Not being bound by theory, since the mineral complex may decrease the risk of mastitis and other illness and positively influence feed utilization, the nutrients and calories present in the animal feed can be more efficiently directed to growth of the animal and milk production. The decreased incidence of mastitis alone would provide for an increase in milk production since mastitis often stops milk production or at the very least renders the milk unfit for consumption, potentially leading to its disposal. Thus, the invention also provides a method for increasing milk production in dairy animals. The invention comprises methods comprising feeding palatable concentrations of the mineral complex of the invention to the dairy animals, and it further comprises feeds containing palatable concentrations of the mineral complex. Preferred dairy animals include cattle, goats, or sheep.

In addition, poultry ingesting the mineral complex in their feed would be expected to remain healthier and in turn show an increase in egg and meat production over poultry not ingesting the mineral complex. Not being bound by theory, because of the decreased incidence of infection and increase in feed efficiency, the nutrients and calories provided in the poultry feed may be more efficiently directed to growth of the animal and egg production. Thus, the present invention provides a method for increasing egg production in poultry. The method of the present invention comprises feeding the mineral complex as described herein to the poultry in palatable concentrations and feeds containing palatable concentrations of the mineral complex for feeding to poultry. The presence of the mineral complex in the bird's digestive tract provides for an increase in egg production over the egg production in poultry not ingesting the mineral complex. In addition, the mineral complex may be effective against Listeria organisms, which can be a significant cause of contamination in poultry production. By inhibiting the growth of Listeria sp. in the digestive tracts of poultry, the mineral complex of the invention would help to decrease or prevent the contamination of poultry carcasses during processing.

The following examples further illustrate the invention but, of course, should not be construed as in any way limiting its scope.

### EXAMPLE 1

This example demonstrates the chemical analysis of several different mineral complexes.

Two mineral complexes (complex 1 and complex 2) were obtained from a natural mineral source and crushed/milled to a particle size of 200 to 400 mesh (a fine dust). A chemical analysis was used to determine the components of each mineral complex, as set forth in Table 3. A third mineral complex (complex 3) was prepared by mixing 95 wt. % of complex 1 with 5 wt. % of complex 2. A fourth mineral complex (complex 4) was prepared by mixing 85 wt. % of complex 1 with 15 wt. % of complex 2.

TABLE 3

| | Complex 1 | | Complex 2 | | Complex 3 | | Complex 4 | |
|---|---|---|---|---|---|---|---|---|
| SiO₂ | 47.6% | | 65.9% | | 48.52% | | 50.35% | |
| Al₂O₃ | 14.75% | | 11.5% | | 14.59% | | 14.26% | |
| Fe₂O₃ | 13.6% | | 1.41% | | 12.99% | | 11.77% | |
| CaO | 9.01% | | 3.75% | | 8.75% | | 8.22% | |
| MgO | 6.36% | | 0.81% | | 6.08% | | 5.53% | |
| Na₂O | 2.6% | | 2.11% | | 2.58% | | 2.53% | |
| K₂O | 0.72% | | 5.25% | | 0.95% | | 1.40% | |
| Cr₂O₃ | 0.02% | | <0.01% | | 0.02% | | 0.02% | |
| TiO₂ | 1.59% | | 0.2% | | 1.52% | | 1.38% | |
| MnO | 0.19% | | 0.02% | | 0.18% | | 0.16% | |
| P₂O₅ | 0.2% | | 0.15% | | 0.20% | | 0.19% | |
| SrO | 0.03% | | 0.03% | | 0.03% | | 0.03% | |
| BaO | 0.02% | | 0.09% | | 0.02% | | 0.03% | |
| C | 0.04% | | 0.62% | | 0.07% | | 0.13% | |
| S | <0.01% | | 0.025% | | <0.01% | | <0.01% | |
| Ag | <1 | ppm | <1 | ppm | <1 | ppm | <1 | ppm |
| Ba | 199 | ppm | 450 | ppm | 211.55 | ppm | 236.65 | ppm |
| Ce | 32 | ppm | 220 | ppm | 41.4 | ppm | 60.20 | ppm |
| Co | 58 | ppm | 21 | ppm | 56.15 | ppm | 52.45 | ppm |
| Cr | 170 | ppm | 6 | ppm | 161.80 | ppm | 145.40 | ppm |
| Cs | 0.72 | ppm | 22 | ppm | 1.78 | ppm | 3.91 | ppm |
| Cu | 190 | ppm | 12 | ppm | 181.10 | ppm | 163.30 | ppm |
| Dy | 5.63 | ppm | 2.5 | ppm | 5.47 | ppm | 5.16 | ppm |
| Er | 3.21 | ppm | 1.7 | ppm | 3.13 | ppm | 2.98 | ppm |
| Eu | 1.79 | ppm | 3.7 | ppm | 1.89 | ppm | 2.08 | ppm |
| Ga | 24.2 | ppm | 15 | ppm | 23.74 | ppm | 22.82 | ppm |
| Gd | 5.56 | ppm | 3.7 | ppm | 5.47 | ppm | 5.28 | ppm |
| Hf | 3.9 | ppm | 21 | ppm | 4.76 | ppm | 6.47 | ppm |
| Ho | 1.06 | ppm | 0.6 | ppm | 1.04 | ppm | 0.99 | ppm |
| La | 14.6 | ppm | 220 | ppm | 24.87 | ppm | 45.41 | ppm |
| Lu | 0.43 | ppm | 0.5 | ppm | 0.43 | ppm | 0.44 | ppm |
| Mo | 3 | ppm | 12.5 | ppm | 3.48 | ppm | 4.43 | ppm |
| Nb | 8.6 | ppm | 40 | ppm | 10.17 | ppm | 13.31 | ppm |
| Nd | 20.6 | ppm | 5 | ppm | 19.82 | ppm | 18.26 | ppm |
| Ni | 105 | ppm | 2.5 | ppm | 99.88 | ppm | 89.63 | ppm |
| Pb | 5 | ppm | 6 | ppm | 5.05 | ppm | 5.15 | ppm |
| Pr | 4.71 | ppm | 27 | ppm | 5.82 | ppm | 8.05 | ppm |
| Rb | 17.2 | ppm | 325 | ppm | 32.59 | ppm | 63.37 | ppm |
| Sm | 4.9 | ppm | 6.2 | ppm | 4.97 | ppm | 5.10 | ppm |
| Sn | 1 | ppm | 3 | ppm | 1.10 | ppm | 1.30 | ppm |
| Sr | 244 | ppm | 380 | ppm | 250.80 | ppm | 264.40 | ppm |
| Ta | 0.6 | ppm | 2.8 | ppm | 0.71 | ppm | 0.93 | ppm |
| Tb | 0.89 | ppm | 0.8 | ppm | 0.89 | ppm | 0.88 | ppm |
| Th | 1.73 | ppm | 180 | ppm | 10.64 | ppm | 28.47 | ppm |
| Tl | <0.5 | ppm | 6 | ppm | <0.78 | ppm | <1.33 | ppm |
| Tm | 0.45 | ppm | 0.6 | ppm | 0.46 | ppm | 0.47 | ppm |
| U | 0.48 | ppm | 6 | ppm | 0.76 | ppm | 1.31 | ppm |
| V | 271 | ppm | 7.8 | ppm | 257.84 | ppm | 231.52 | ppm |
| W | 1 | ppm | 26 | ppm | 2.25 | ppm | 4.75 | ppm |
| Y | 29.2 | ppm | 23 | ppm | 28.89 | ppm | 28.27 | ppm |
| Yb | 2.93 | ppm | 1.4 | ppm | 2.85 | ppm | 2.70 | ppm |
| Zn | 135 | ppm | 64 | ppm | 131.45 | ppm | 124.35 | ppm |
| Zr | 138 | ppm | 63 | ppm | 134.25 | ppm | 126.75 | ppm |
| As | 0.8 | ppm | 1.1 | ppm | 0.82 | ppm | 0.85 | ppm |
| Bi | 0.01 | ppm | 3.5 | ppm | 0.18 | ppm | 0.53 | ppm |
| Hg | <0.005 | ppm | 0.01 | ppm | <0.005 | ppm | <0.006 | ppm |
| Sb | 0.06 | ppm | 0.4 | ppm | 0.08 | ppm | 0.11 | ppm |
| Se | 0.6 | ppm | 0.7 | ppm | 0.61 | ppm | 0.62 | ppm |
| Te | <0.01 | ppm | 0.022 | ppm | <0.01 | ppm | <0.01 | ppm |

| | | | | |
|---|---|---|---|---|
| LOI | 2.76% | 6.45% | 2.94% | 3.31% |

The silicon dioxide content of mineral complex 2 falls outside of the scope of the mineral complex described herein. However, mineral complex 2 was successfully combined with mineral complex 1 to prepare mineral complexes 3 and 4. Mineral complexes 1, 3, and 4 fall within the scope of the invention described herein. Additional mineral complexes falling within the scope of the invention described herein can be prepared using mineral complexes 1 and 2. This example demonstrates the specific components of three exemplary mineral complexes of the invention that may be administered to animals or used to supplement any prepared or formulated animal feed.

### EXAMPLE 2

This example demonstrates that an animal feed supplemented with the mineral complexes described herein increased weight gain of pigs as compared to the same animal feed without the mineral complex.

A large-scale trial with 9000 pigs from wean to nursery exit was designed to test the weight gain of pigs fed an animal feed supplemented with three different mineral complexes as compared to the weight gain of pigs fed the same animal feed without the mineral complex. The nursery phase was 8 weeks. 4 groups each of 2250 pigs cycled through, with staggered arrival and departure of 1 group per week (2 truckloads) as is normal practice. The trial schedule was 12 weeks total.

Prior to arrival at the facility each truck was weighed via DOT scales. At arrival to the facility each group was randomly split to preclude genetic predisposition or exposure to sow-related pathogens. Each truck carried approximately 1150 pigs. The pigs were antibiotic-free at the time of arrival. However, a small number of pigs had been medicated at the sow unit prior to shipping, and were identified with ear tags as per federal regulation. Medicated pigs were evenly distributed in control and test groups at the time of arrival sorting. Starting weights were determined by an even split of the truck load weight. Due to the large number of pigs in the study this method was necessitated.

The groups of arriving pigs were randomly split between paired control and test rooms into a total of 8 rooms (e.g., test room 1 and control room 2, test room 3, and control room 4, etc.). Of the 8 rooms (barns) within the facility—4 control rooms and 4 test rooms—the split weight method was accurate with the exception of room 1 (test) and room 2 (control). In this instance there was a recognizable size/weight difference of 10% (1.32 lbs) favoring room 2 (control).

Nursery pigs are typically fed a 4 phase diet, wherein each phase consists of differing nutritional content. The animal feed used in this study for each phase of the diet is shown in Table 4.

TABLE 4

| | Phase 1 | Phase 2 | Phase 3 | Phase 4 |
|---|---|---|---|---|
| Ingredient | (lbs) | (lbs) | (lbs) | (lbs) |
| Soy Meal 47.5% CP | 688.50 | 601.83 | 743.31 | 740.87 |
| 90% DM | | | | |
| Corn SH 8.5% CP 90% DM | 547.04 | 743.98 | 876.62 | 1074.52 |
| Whey | 400.00 | 250.00 | 50.00 | — |
| Choice White | 96.03 | 95.77 | 100.00 | 99.17 |
| Oats 901330 | 75.00 | 85.00 | 85.00 | — |
| Fish Meal | 75.00 | 75.00 | 75.00 | — |
| Dried Select Egg | 50.00 | 50.00 | — | — |
| Mono Cal 21% PH | 32.77 | 56.29 | 35.28 | 27.52 |
| Lactose 305 | 16.66 | 16.66 | — | — |
| Zinc Oxide | 8.52 | 8.51 | 8.51 | 8.51 |
| BlackJack Sow Start 5 | 6.25 | 5.00 | 6.29 | 6.25 |
| VTM | | | | |
| Lysine | 3.38 | 5.94 | — | — |
| Copper Sulf 25 | 0.86 | 0.89 | 1.00 | 0.37 |
| Soy Hulls | — | 5.12 | — | 20.00 |
| CA Carb | — | — | 17.73 | 15.76 |
| Salt (White) | — | — | 1.27 | 5.03 |
| Phytase 1200 | — | — | — | 2.00 |
| Total (lbs) | 2000.00 | 2000.00 | 2000.00 | 2000.00 |

Approximately 4500 antibiotic-free nursery pigs received standard animal feed, as set forth in Table 4, at each of the 4 phases of the diet (control diet). These pigs were designated as control rooms 2, 4, 6, and 8.

Approximately 1125 antibiotic-free nursery pigs received standard animal feed, as described in Table 4, supplemented with 1 wt. % of mineral complex 1 as described in Example 1 (see Table 3). In order to supplement the animal feed described in Table 4 with 1 wt. % of mineral complex 1, 20 lbs of mineral complex 1 was added to 1980 lbs of the prepared animal feed. The supplemented animal feed was then mixed to ensure an even distribution of the mineral complex. These pigs were designated as test room 1.

Approximately 1125 antibiotic-free nursery pigs received standard animal feed, as described in Table 4, supplemented with 1 wt. % of mineral complex 3 as described in Example 1 (see Table 3). In order to supplement the animal feed described in Table 4 with 1 wt. % of mineral complex 3, 20 lbs of mineral complex 3 was added to 1980 lbs of the prepared animal feed. The supplemented animal feed was then mixed to ensure an even distribution of the mineral complex. These pigs were designated as test room 3.

Approximately 1125 antibiotic-free nursery pigs received standard animal feed, as described in Table 4, supplemented with 1 wt. % of mineral complex 4 as described in Example 1 (see Table 3). In order to supplement the animal feed described in Table 4 with 1 wt. % of mineral complex 4, 20 lbs of mineral complex 4 was added to 1980 lbs of the prepared animal feed. The supplemented animal feed was then mixed to ensure an even distribution of the mineral complex. These pigs were designated as test room 5.

Approximately 1125 antibiotic-free nursery pigs received standard animal feed, as described in Table 4, supplemented with 2 wt. % of mineral complex 1 as described in Example 1 (see Table 3). In order to supplement the animal feed described in Table 4 with 2 wt. % of mineral complex 1, 40 lbs of mineral complex 1 was added to 1960 lbs of the prepared animal feed. The supplemented animal feed was then mixed to ensure an even distribution of the mineral complex. These pigs were designated as test room 7.

Samples of the control and test groups (4 pens from each of the 4 control groups and 4 pens from each of the 4 test groups) were weighed at each diet phase transition to assist in identifying growth/health characteristics per diet phase. Each phase was timed and monitored via total volume of feed consumed per group (per room) such that the rate of consumption could be compared between all control and test groups.

The average weight at exits of the pigs in each of the paired control and test rooms is shown in Table 5.

TABLE 5

| | Average Total | Average Daily | Average Daily |
|---|---|---|---|
| | Gain per Pig | Gain per Pig | Feed Consumption |
| | (lbs) | (lbs) | (lbs) |
| Test Room 1 | 41.61 | 0.832 | 1.37 |
| Control Room 2 | 39.41 | 0.821 | 1.37 |
| Test Room 3 | 41.05 | 0.838 | 1.29 |
| Control Room 4 | 39.43 | 0.813 | 1.25 |
| Test Room 5 | 43.25 | 0.874 | 1.34 |
| Control Room 6 | 39.02 | 0.765 | 1.34 |
| Test Room 7 | 40.35 | 0.858 | 1.36 |
| Control Room 8 | 37.54 | 0.743 | 1.39 |

The data presented in Table 5 demonstrate that the pigs in each of the test rooms, wherein the pigs received animal feed supplemented with mineral complex, consistently gained more weight as compared to the pigs in the paired control room. The data presented in Table 5 also demonstrate that this difference in weight gain was not a result of increased feed consumption because the average daily feed consumption was the same for each of the paired control and test rooms. The combined data for all of the control rooms demonstrates that the average weight gain at exit per pig receiving the control diet was 38.85 lbs. In comparison, the combined data for all of test rooms demonstrates that the average weight gain at exit of each pig receiving the test diet comprising the mineral complex was 41.57 lbs. These results demonstrate that pigs receiving animal feed supplemented with either 1 wt. % or 2 wt. % of mineral complex gained an average of 2.72 lbs more than pigs receiving standard animal feed without mineral complex.

The results of this trial demonstrate that the supplemented animal feed, which included either 1 wt. % or 2 wt. % pound for pound replacement of the animal feed mix with mineral complexes of the invention, increased the average weight gain per test nursery pig as compared to the average weight gain per control nursery pig. Therefore, the results of the trial presented in this example demonstrate that the supplemented animal feed of the invention effectively increases the weight gain of animals as compared to the weight gain of animals receiving standard animal feed.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A supplemented animal feed comprising an edible animal feed and a mineral complex, wherein the mineral complex comprises about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO.

2. The supplemented animal feed of claim 1, wherein the mineral complex further comprises Al₂O₃, wherein the Al₂O₃ is present at less than 16 wt. %.

3. The supplemented animal feed of claim 1, wherein the mineral complex further comprises about 12 wt. % to about 15 wt. % Al₂O₃.

4. The supplemented animal feed of claim 1, wherein the mineral complex further comprises about 1 wt. % to about 4 wt. % Na₂O.

5. The supplemented animal feed of claim 1, wherein the mineral complex further comprises at least one rare earth element selected from the group consisting of scandium, yttrium, lanthanum, cerium, praseodymium, neodymium, promethium, samarium, europium, gadolinium, terbium, dysprosium, holmium, erbium, thulium, ytterbium, and lutetium.

6. The supplemented animal feed of claim 1, wherein the mineral complex further comprises at least one of the following: K₂O, Cr₂O₃, TiO₂, MnO, P₂O₅, SrO, or BaO.

7. The supplemented animal feed of claim 1, wherein mineral complex is milled prior to addition to the animal feed.

8. The supplemented animal feed of claim 1, wherein the supplemented animal feed is pelletized, said pellets comprising the edible animal feed and the mineral complex.

9. The supplemented animal feed of claim 1, wherein the supplemented animal feed is extruded, said extruded animal feed comprising the edible animal feed and the mineral complex.

10. The supplemented animal feed of claim 1, wherein the supplemented animal feed comprises an admixture of the edible animal feed and the mineral complex.

11. The supplemented animal feed of claim 1, wherein the mineral complex is present in the supplemented animal feed in an amount ranging from about 0.1 lbs to about 5 lbs of mineral complex per 100 pounds of supplemented animal feed.

12. The supplemented animal feed of claim 11, wherein the mineral complex is present in the supplemented animal feed in an amount ranging from about 1 lb to about 2 lbs of mineral complex per 100 pounds of the supplemented animal feed.

13. A process for preparing a supplemented animal feed comprising:
(a) providing a prepared or formulated edible animal feed; and
(b) adding to the edible animal feed a mineral complex comprising about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO.

14. The process of claim 13, wherein about 0.1 lbs to about 5 lbs of the mineral complex is added per 100 pounds of supplemented animal feed.

15. The process of claim 14, wherein about 1 lbs to about 2 lbs of mineral complex is added per 100 pounds of the supplemented animal feed.

16. A process for preparing a supplemented animal feed comprising:
(a) providing the ingredients for an animal feed;
(b) adding to the animal feed ingredients a mineral complex comprising about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO; and
(c) processing the animal feed,
wherein the resulting final form of the animal feed after processing comprises the mineral complex.

17. The process of claim 16, wherein about 0.1 lbs to about 5 lbs of the mineral complex is added per 100 pounds of animal feed ingredients.

18. The process of claim 17, wherein about 1 lbs to about 2 lbs of mineral complex is added per 100 pounds of animal feed ingredients.

19. An animal feed composition comprising a mineral complex and a binder, wherein the mineral complex comprises about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO.

20. The animal feed composition of claim 19, wherein the binder is selected from the group consisting of molasses, gelatin, urea formaldehyde, humic, carboxymethylcellulose, gum arabic, guar gum, and lignin sulfonate.

21. The animal feed composition of claim 19, wherein the composition comprises from about 0.1 wt. % to about 50 wt. % binder.

22. The animal feed composition of claim 21, wherein the composition comprises from about 0.5 wt. % to about 2.5 wt. % binder.

23. A method for supplementing the diet of an animal comprising administering to an animal in need thereof an effective amount of a mineral complex comprising about 40 wt. % to about 60 wt. % SiO₂, about 6 wt. % to about 16 wt. % Fe₂O₃, about 4 wt. % to about 12 wt. %. CaO, and about 2 wt. % to about 8 wt. % MgO.

24. The supplemented animal feed of claim 1, wherein the mineral complex comprises about 4 wt. % to about 8 wt. % MgO.

25. The supplemented animal feed of claim 1, wherein the mineral complex comprises about 7 wt. % to about 10 wt. %. CaO.

26. The supplemented animal feed of claim 1, wherein the mineral complex comprises about 0.0001 ppm to 1.31 ppm of uranium.
